(19) United States
(12) Patent Application Publication
(10) Pub. No.: US 2026/0232584 A1
(43) Pub. Date: Aug. 13, 2026

(54) TOPICAL PAIN RELIEF COMPOSITION

(71) Applicant: ACHELON, INC., Redondo Beach, CA (US)

(72) Inventor: Manju Chacko DAWKINS, Redondo Beach, CA (US)

(21) Appl. No.: 19/160,719

(22) PCT Filed: Feb. 29, 2024
(86) PCT No.: PCT/US2024/017899

§ 371 Date: Aug. 28, 2025

Related U.S. Application Data
(60) Provisional application No. 63/487,751, filed on Mar. 1, 2023.

(51) Int. Cl. A61K 9/06; A61K 36/28; A61K 36/67; A61K 36/9066; A61K 47/08

(52) U.S. Cl. CPC A61K 9/06; A61K 36/28; A61K 36/67; A61K 36/9066; A61K 47/08

(57) ABSTRACT

The present invention relates to topical formulations, creams, serums, kits and adhesive patches including Arnica, black pepper, and turmeric. Also provided are methods for decreasing soreness, pain, bruising, and/or swelling in a subject by administering topical formulations and adhesive patches.

### CROSS REFERENCE TO RELATED APPLICATIONS

This application claims the benefit of priority of U.S. Provisional Patent Application No. 63/487,751, filed Mar. 1, 2023, the entire contents of which is incorporated by reference herein.

### BACKGROUND

Unfortunately, millions of people are experiencing unnecessary pain associated with needle sticks. Vaccinations, venipuncture, and other sharps-based procedures can be painful and fear-inducing experiences. Ultimately, this pain and fear may lead people to avoid important preventative health procedures. In fact in 2015, the World Health Organization announced that “pain mitigation is an important strategy to counter vaccine hesitancy.” The rate of preventable diseases increases as vaccination rates decrease, which negatively affects young and susceptible populations. Even compliant patients, who receive recommended vaccinations regardless of the pain, would likely prefer to feel less pain if a simple solution existed.

### SUMMARY

### Over-the-Counter Products

Most post-needle stick pain goes untreated, as only a bandage is applied to the needle stick site. There are over-the-counter local analgesic products which contain lidocaine available, but most people are not aware of the benefit to post-needle procedure pain. Subjects will typically take oral over-the-counter pain relievers, such as acetaminophen, ibuprofen, aspirin, or naproxen. However, these over-the-counter products are not typically formulated to specifically address needle sticks and potentially have several drawbacks:
1) Over-the-counter lidocaine products are not typically indicated for the treatment of needle stick pain.
   1a) The majority of over-the-counter lidocaine patch products are indicated to treat back, neck, and shoulder pain.
   1b) Other over-the-counter lidocaine products are indicated to provide temporary relief of pain and itching associated with minor burns, sunburn, minor cuts, insect bites, or minor skin irritation.
2) Systemic over-the-counter products have a number of serious side effects. Excessive acetaminophen use has been associated with liver damage. NSAIDS, including ibuprofen, aspirin, and naproxen can cause stomach irritation and be toxic to the kidneys.

### Prescription Products

There are other topical anesthetic products available to patients via prescription. To the Applicant's knowledge, in the prescription market, there are few available products that specifically attempt to address needle stick pain. These products include a topical product commonly prescribed by physicians to prevent needle stick pain—EMLA. EMLA is not often prescribed for post-needle procedure pain. In general, as discussed below, there are meaningful challenges for the average consumer to obtain prescriptions. There are also significant potential drawbacks to the popular prescriptions used for needle sticks, specifically EMLA.

### General Prescription Drawbacks

1) Prescriptions add time for families who need to fill the prescriptions at a preceding appointment that differs from the procedure date.
2) Prescriptions add time for physicians who need to write the prescription at a preceding appointment that differs from the procedure date.
3) Individuals and families could potentially pay a high copay or drug cost.
4) The average consumer lacks the confidence and knowledge of how to adapt existing products with a different indication and apply that to pre-needle procedures.

### EMLA Prescription

EMLA, a eutectic mixture of 2.5% lidocaine and 2.5% prilocaine, is prescribed commonly by physicians, off-label in an attempt to prevent pain associated with needle sticks. However, this product presents several potential drawbacks:
1) The medication is formulated to have extended release into the skin over a period of 12 hours.
2) EMLA contains a secondary drug, prilocaine, which increases the risk of methemoglobinemia, a potentially fatal disease.
3) Although EMLA exists in a cream and patch form, it is almost always dispensed in the cream form.
4) EMLA must be left on for more than an hour in order to be effective.
5) The cream must be occluded in order to be effective. Most doctors advise their patients to use plastic food wrap to occlude the cream. For most patients, this process is both messy and cumbersome. There are medical dressings that can be purchased separately, but this increases costs to the patient and adds an additional step in the application process.
6) Instructions for use on these products are not specific to needle procedures. Since there is no true indication for this use, there are no clear instructions for the patient regarding dosage, duration, and location.
   6a) Dosage—Due to lack of instruction, patients often do not use enough of EMLA. This makes the treatment ineffective and causes the patient to take on the added risk of using medication without experiencing the benefit of reduced pain.
   6b) Duration—Due to lack of instruction, patients often do not leave EMLA on their skin long enough. This makes the treatment ineffective and causes the patient to take on the added risk of using medication without experiencing the benefit of reduced pain.
   6c) Location—If a patient takes it upon themselves to use a topical cream, the site of treated skin is often lost once the medication is wiped away. The medication also often migrates to a different area than intended. For example, if the patient occludes the cream with plastic food wrap, there is often sliding under clothing and the intended site is inadequately treated. The treatment can potentially be ineffective and causes the patient to take on the added risk of using medication without experiencing the benefit of reduced pain.
7) Individuals and families could potentially pay a high copay or drug cost.
8) The average consumer lacks the confidence and knowledge to adapt existing products with a different indication and apply that to needle procedures.

Safe topical pain relief solved for an important unmet need. Therefore, provided for herein in several embodiments are topical formulations, adhesive patches, and methods of administering topical formulations including turmeric, Arnica, and black pepper. According to several embodiments, the topical formulations that are provided for herein reduce a subject's soreness, pain, bruising, and/or swelling of the skin, subcutaneous tissue, and/or muscles at a needle stick site.

In some embodiments, the topical formulation comprises Arnica present in an amount of 10% to 25% or about 10% to about 25% w/w; black pepper present in an amount of 0.5% to 7% or about 0.5% to about 7% w/w; and turmeric present in an amount 5% to 16% or about 5% to about 16% w/w.

In some embodiments, the Arnica is in the form of in the form of Arnica extract, ground Arnica, Arnica powder, Arnica essential oil, Arnica distillate, Arnica hydrosol, Arnica ointment, Arnica tincture, Arnica cream, Arnica liniment, Arnica salve and/or Arnica gel. In some embodiments, the Arnica is in the form of Arnica extract. In some embodiments, the black pepper is in the form of black pepper extract, ground black pepper, black pepper powder, black pepper essential oil, black pepper distillate, black pepper hydrosol, black pepper ointment, black pepper tincture, black pepper cream, black pepper liniment, black pepper salve and/or black pepper gel. In some embodiments, the black pepper is in the form of black pepper extract. In some embodiments, the black pepper active ingredient is in the form piperine. In some embodiments, the turmeric is in the form of in the form of turmeric root extract, ground turmeric, turmeric powder, turmeric essential oil, turmeric distillate, turmeric hydrosol, turmeric ointment, turmeric tincture, turmeric cream, turmeric liniment, turmeric salve and/or turmeric gel. In some embodiments, the turmeric is in the form of turmeric extract.

In some embodiments, the topical formulation also includes a preservative. In some embodiments, the preservative comprises Coenzyme Q10 (CoQ10). In some embodiments, the CoQ10 is present in an amount 0.05% to 1% or about 0.05% to about 1% w/w.

In some embodiments, the Arnica is present in an amount of 13-15% (e.g., 14.6%) or is about 13-15% (e.g., 14.6%) w/w, the black pepper is present in an amount of 0.5-1.5% (e.g., 1.0%) or is about 0.5-1.5% (e.g., about 1.0% w/w), and the turmeric is present in an amount of 7.5-9.0% (e.g., 8.1%) or is about 7.5-9.0% (e.g., about 8.1%) w/w. In several embodiments, when present, CoQ10 is present in an amount of 0.1-0.5% (e.g., 0.3%) or is about 0.1-0.5% (e.g., about 0.3%) w/w.

In some embodiments, the Arnica is present in an amount of 22-25% (e.g., 23.02%) or is about 22-25% (e.g., about 23.02% w/w), the black pepper is present in an amount of 0.5-1.5% (e.g., 0.89%) or is about 0.5-1.5% (e.g., about 0.89%) w/w, and the turmeric extract is present in an amount of 6.5-8% (e.g., 7.3%) or is about 6.5-8% (e.g., about 7.3%) w/w. In several embodiments, when present, CoQ10 is present in an amount of 0.1-0.5% (e.g., 0.26%) or is about 0.1-0.5% (e.g., about 0.26%) w/w.

In some embodiments, the topical formulation comprises at least one plant product selected from group consisting of Zanthoxylum, clove, thyme, Piper betle, peppermint, cinchona, datura, jasmine, California sagebrush, lavender, paracress, sterculia, fennel, nutmeg, cinnamon, basil, and chili pepper. In some embodiments, the topical formulation further comprises linalool.

In some embodiments, the topical formulation does not comprise a chemical anesthetic. In some embodiments, the topical formulation does not comprise lidocaine, tetracaine, or benzocaine. In some embodiments, the topical formulation does not comprise menthol.

In some embodiments, the topical formulation also includes a pressure sensitive adhesive. In some embodiments, the pressure sensitive adhesive is present in an amount of 48% to 84.5% or about 48% to 84.5% w/w. In some embodiments, the pressure sensitive adhesive is present in an amount of 76.0% or about 76.0% w/w.

In some embodiments, a cream for decreasing soreness, pain, bruising, and/or swelling in a subject comprises a topical formulation of Arnica, black pepper, and turmeric. In some embodiments, the cream also includes a preservative. In some embodiments, the preservative comprises Coenzyme Q10 (CoQ10). In some embodiments, the cream includes any one of the topical formulations disclosed herein. In some embodiments, the soreness, pain, bruising, and/or swelling is localized to an anatomical region of the subject. In some embodiments, the anatomical region comprises skin, nail, mucosa, eye, muscle, and/or bone. In some embodiments, the soreness, pain, bruising, and/or swelling comprises an ecchymosis. In some embodiments, the soreness, pain, bruising, and/or swelling is related to a needle procedure, a surgical procedure, an injury, an insect bite, arthritis, and/or radiation therapy. In some embodiments, the subject is an animal, optionally a human.

In some embodiments, a serum for decreasing soreness, pain, bruising, and/or swelling in a subject comprises a topical formulation of Arnica, black pepper, and turmeric. In some embodiments, the serum also includes a preservative. In some embodiments, the preservative comprises Coenzyme Q10 (CoQ10). In some embodiments, the serum includes any one of the topical formulations disclosed herein. In some embodiments, the soreness, pain, bruising, and/or swelling is localized to an anatomical region of the subject. In some embodiments, the anatomical region comprises skin, nail, mucosa, eye, muscle, and/or bone. In some embodiments, the soreness, pain, bruising, and/or swelling comprises an ecchymosis. In some embodiments, the soreness, pain, bruising, and/or swelling is related to a needle procedure, a surgical procedure, an injury, an insect bite, arthritis, and/or radiation therapy. In some embodiments, the subject is an animal, optionally a human.

In some embodiments, an adhesive patch for decreasing soreness, pain, bruising, and/or swelling in a subject comprises a first superior surface; a second inferior surface configured for application onto the subject's skin, the second inferior surface comprising a topical formulation, wherein the topical formulation comprises Arnica, black pepper, and turmeric. In some embodiments, the second inferior surface of the adhesive patch further comprises a sterile bandage. In some embodiments, the sterile bandage is located at the center of the second inferior surface of the adhesive patch. In some embodiments, the adhesive patch also includes a preservative. In some embodiments, the preservative comprises Coenzyme Q10 (CoQ10). In some embodiments, the adhesive patch also includes a pressure sensitive adhesive. In some embodiments, the adhesive patch includes any one of the topical formulations disclosed herein. In some embodiments, the adhesive patch is contained within sterile packaging. In some embodiments, the soreness, pain, bruising, and/or swelling is localized to an anatomical region of the subject. In some embodiments, the anatomical region comprises skin, nail, mucosa, eye, muscle, and/or bone. In some embodiments, the soreness, pain, bruising, and/or swelling comprises an ecchymosis. In some embodiments, the soreness, pain, bruising, and/or swelling is related to a needle procedure, a surgical procedure, an injury, an insect bite, arthritis, and/or radiation therapy. In some embodiments, the subject is an animal, optionally a human.

In some embodiments, a kit for use in decreasing soreness, pain, bruising, and/or swelling in a subject, comprises a predetermined dose of a topical formulation comprising Arnica, black pepper, and turmeric; and an adhesive dressing. In some embodiments, the kit also includes a preservative. In some embodiments, the preservative comprises Coenzyme Q10 (CoQ10). In some embodiments, the kit includes any one of the topical formulations disclosed herein. In some embodiments, the soreness, pain, bruising, and/or swelling is localized to an anatomical region of the subject. In some embodiments, the anatomical region comprises skin, nail, mucosa, eye, muscle, and/or bone. In some embodiments, the soreness, pain, bruising, and/or swelling comprises an ecchymosis. In some embodiments, the soreness, pain, bruising, and/or swelling is related to a needle procedure, a surgical procedure, an injury, an insect bite, arthritis, and/or radiation therapy. In some embodiments, the subject is an animal, optionally a human.

In some embodiments, a method for decreasing soreness, pain, bruising, and/or swelling in a subject comprises topical administration to the subject of a therapeutically effective amount of any of the topical formulations disclosed herein. In some embodiments, the topical formulation is administered to the subject for 8-48 hours or about 8-48 hours. In some embodiments, the topical formulation is administered to the subject at or around a site of pain. In some embodiments, the site of pain comprises a needle stick site, an injury, a joint, and/or an insect bite. In some embodiments, the topical formulation is administered at or around the needle stick site, post-needle stick. In some embodiments, the soreness, pain, bruising, and/or swelling is localized to an anatomical region of the subject. In some embodiments, the anatomical region comprises skin, nail, mucosa, eye, muscle, and/or bone. In some embodiments, the soreness, pain, bruising, and/or swelling comprises an ecchymosis. In some embodiments, the soreness, pain, bruising, and/or swelling is related to a needle procedure, a surgical procedure, an injury, an insect bite, arthritis, and/or radiation therapy. In some embodiments, the subject is an animal, optionally a human.

In some embodiments, a method for decreasing soreness, pain, bruising, and/or swelling in a subject comprises applying any of the adhesive patches disclosed herein. In some embodiments, the adhesive patch is applied to the subject for 8-48 hours or about 8-48 hours. In some embodiments, the adhesive patch is applied to the subject at or around a site of pain. In some embodiments, the site of pain comprises a needle stick site, an injury, a joint, and/or an insect bite. In some embodiments, the adhesive patch is applied to at or around the needle stick site, post-needle stick. In some embodiments, the soreness, pain, bruising, and/or swelling is localized to an anatomical region of the subject. In some embodiments, the anatomical region comprises skin, nail, mucosa, eye, muscle, and/or bone. In some embodiments, the soreness, pain, bruising, and/or swelling comprises an ecchymosis. In some embodiments, the soreness, pain, bruising, and/or swelling is related to a needle procedure, a surgical procedure, an injury, an insect bite, arthritis, and/or radiation therapy. In some embodiments, the subject is an animal, optionally a human.

### DETAILED DESCRIPTION

Pain management can be considered a basic human right and pain from needle based procedures and other superficial skin procedures affect an overwhelming number of individuals. Needle phobia negatively impacts medical procedures involving needles such as vaccinations, necessary blood draws, and blood donations. Vaccinations, alone, are the most common painful needle procedure worldwide. The scope of the problem is immense. There is a clear need for an affordable and easy to use product that is broadly available with the specific purpose to address pain from needle based procedures, as is provided for by embodiments disclosed herein. In the over the counter market specifically, there is a noticeable void that can be filled by addressing the drawbacks presented by other products. Lastly, the topical formulations and patch according to embodiments disclosed herein has positive and far reaching implications for families, the medical industry, and global health.

Currently, the medical community is incentivized for achieving high patient satisfaction scores. Members of the medical community constantly seek different approaches that can safely improve and meaningfully impact the patient's health experience. The medical industry would welcome embodiments of the present disclosure at least in part due to its low cost and low risk. Several embodiments of the present disclosure provide families with an accessible, affordable, and user-friendly product that gives them ownership over their own and their children's pain associated with needle sticks.

Embodiments of the present disclosure address certain aspects of major public health issues and increasing health care costs. The number of adults with needle phobia has been vastly underestimated and is not a myth. In fact, needle phobia has been defined by the American Psychiatric Association as a formal medical condition that affects at least 10% of the population according to a 1995 study. A more recent study done in 2012 by Taddio et al. suggests a rate of needle fear closer to 24%. While exact numbers are not known, it is clear that a large and increasing number of people are impacted by fear of needles.

First, individuals who suffer from needle phobia are less likely to receive vaccines. Vaccines help prevent unnecessary and potentially serious illness. In addition, vaccines help protect families and communities and can potentially eliminate diseases worldwide. Vaccine avoidance is not only detrimental to the patient, but also to the broader population and society as a whole. Noncompliance with vaccinations results in decreased herd immunity, which particularly protects young and susceptible populations. Noncompliance also leads to higher rates of preventable, debilitating, and deadly diseases. Vaccinations, alone, are the most common painful needle procedure worldwide with an estimated 12 billion injections given per year. Twenty-four percent of parents and 63% of children report some fear of needles. The same study reports that needle fear is the primary reason for immunization non-compliance for 7% and 8% of parents and children, respectively. In the United States alone, the Centers for Disease Control and Prevention (“CDC”) dictates that every American child should receive a total of 69 doses of 16 vaccines by age 18 in order to comply with recommendations. Of the 69 total doses, 26 doses of 9 different vaccines should be given in a child's first year of life. 48 doses of 14 different vaccines should be given by the age of 5 or 6. The National Association for the Education of Young Children (NAEYC) estimates that there are approximately 21 million children under the age of 5 in the United States. Based on CDC recommended vaccinations in the United States, 100 million shots per year are administered for our children under the age of 6.

Second, blood draws are necessary for the prevention and proper management of disease. For example, if type II diabetes is diagnosed early with a simple blood test, it is treatable by improving diet and exercise. In this instance, patients with type II diabetes can remain healthy for decades. On the other hand, if a patient has been avoiding blood tests, he or she might have a longstanding and untreated case of type II diabetes, where the only option may be to rely on daily insulin injections. Daily insulin injections, ironically, subject the patient to pain that they were likely attempting to avoid. Studies have found that 51% of adults with type 2 diabetes delay starting insulin due to needle fear and 94% feel fear reactions every time they inject. In a worst case scenario, the damage may already be done, and the patient could develop permanent blindness, require an amputation, or fall into a diabetic coma. At a certain point, it may be too late for intervention at all. Needle fear is likely preventing a large portion of the population form receiving appropriate medical care, which also increases rates and severity of disease.

Third, those who dislike needles have almost surely avoided donating blood. The Red Cross has reported severe blood shortages as recent as September 2016. In addition, the law of supply and demand dictates that blood scarcity results in increased cost of blood. Therefore, these shortages result in rising costs in health care. Even though an estimated 38% of the U.S. population is eligible to donate blood at any given time, less than 10% of the eligible population actually do so each year.

In several embodiments, the devices disclosed herein allow needle-based procedures to be more tolerable. For instance, the simple, cost-effective patch has the potential to make a significant economic and health impact. These combined factors contribute to increased health care costs globally. They also give a sense of how needle phobia and needle fear can negatively impact the broader public health system and economy.

Disclosed herein are embodiments of topical formulations, adhesive patches, and methods of administering topical formulations including turmeric, Arnica, and black pepper. Turmeric, Arnica, and black pepper are typically found in nature on different continents. In combination, these three compounds potentiate, in a synergistic manner, each other's therapeutic effects in terms of a reducing a subject's soreness, pain, bruising, and/or swelling of the skin, subcutaneous tissue, and/or muscles at a needle stick site.

### Terms

The terms “a”, “an”, or “the” as used herein not only include aspects with one member, but also include aspects with more than one member.

The term “about” as used herein to modify a numerical value indicates a defined range around that value. If “X” were the value, “about X” would generally indicate a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X”. When the quantity “X” only includes whole-integer values (e.g., “X carbons”), “about X” indicates from (X−1) to (X+1). In this case, “about X” as used herein specifically indicates at least the values X, X−1, and X+1. When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11%” is equivalent to “about 7%, about 9%, or about 11%.”

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

“Agent” as used herein indicates a compound or mixture of compounds that, when added to a composition, tend to produce a particular effect on the said composition's properties or performance. For example, a composition comprising a thickening agent is likely to be more viscous than an otherwise identical comparative composition that lacks the thickening agent; a composition comprising an active agent is more likely to provide a beneficial effect in the subject to which the composition is administered that otherwise.

The terms “chassis”, “vehicle” and “base formulation” as used interchangeably herein are equivalent terms that include a plurality of solvents or other excipients that comprise the bulk of a formulation, into which one or more active agents or additional agents might be introduced.

As used herein, the phrase “effective amount” or “effective dose” means an amount sufficient to achieve the desired result and accordingly will depend on the ingredient and its desired result. Nonetheless, once the desired effect is known, determining the effective amount is within the skill of a person skilled in the art.

“Formulation,” “pharmaceutical composition,” and “composition” as used interchangeably herein are equivalent terms referring to a composition of matter for administration to a subject.

“Gel” as used herein means a semisolid system consisting of either a suspension made up of small inorganic particles or large organic molecules interpenetrated by a liquid. The viscosity of a gel may be such that it is either flowable or non-flowable.

The prefix “micro” as used herein can be alternatively abbreviated as “u” or “u.” For example, micrograms are typically abbreviated as μg, but can alternatively be abbreviated as “ug”.

The term “onset of action” as used herein means the amount of time it takes for the effects of an active to become noticeable after administration in a formulation to the skin.

The term “or” as used herein should in general be construed non-exclusively. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

As used herein, the term “pain from needle sticks” shall be given its ordinary meaning and also refers to pain associated insertion of a needle of any gauge and length for the purpose of (1) Injecting medication intramuscularly, subcutaneously, and intradermally; (2) Venipuncture, which as used herein shall be given its ordinary meaning, and also refers to the puncture of a vein as part of a medical procedure, typically to withdraw a blood sample or for an intravenous injection; and (3) Finger-sticks, which as used herein, shall be given its ordinary meaning and also refers to a procedure in which a finger is pricked with a blood lancet or pricking needle to obtain a small quantity of capillary blood for testing.

As used herein, the term “pain from a sharps procedure” shall be given its ordinary meaning and also refer to a procedure using a sharp instrument, e.g., a needle, a scalpel, etc. As used herein, the term “pain from a superficial skin procedure” shall be given its ordinary meaning and also refers to but is not limited to shave biopsies, punch biopsies, scissor excisions, incisions and drainage, acne surgery, electrodessication, curettage, cryotherapy, and laser treatment. As used herein, the term “pain from tattooing, electrolysis, laser, and piercing” shall be given its ordinary meaning and also refer to but is not limited pain from tattooing, electrolysis, laser, and piercing.

As used herein, the term “pain-relieving amount” shall be given its ordinary meaning and also refers to the amount of any of the active ingredients provided for herein and that result in the reduction, decrease, amelioration or elimination of pain.

The term “pharmaceutically acceptable” means compatible with the treatment of animals, and in particular, humans.

The term “pharmaceutically acceptable salt” means a pharmaceutically acceptable acid addition salt or a pharmaceutically acceptable base addition salt. The formation of a desired compound salt is achieved using standard techniques. For example, the neutral compound is treated with an acid or base in a suitable solvent and the formed salt is isolated by filtration, extraction, or by any other suitable method.

As used herein, the term “subject” refers to all animals including humans. Examples of patients or subjects include humans, cows, dogs, cats, goats, sheep, and pigs. As used herein, the term “subject” may also be referred to as a “patient.”

“Superficial delivery” as used herein means delivery of an agent to the skin exterior surface only.

As used herein, the term “therapeutically effective amount” shall be given its ordinary meaning and also refers to that amount of any active ingredient, e.g., turmeric, black pepper, Arnica, which provides a therapeutic or beneficial effect for a given condition and administration regimen to a subject. Depending on the embodiment, the concentration of the substance is selected so as to exert its pharmaceutical effect, while in several embodiments, avoiding significant side effects to a subject. The effective amount may vary, depending on the embodiment on, for example, one or more of the treatment site, the age and physical condition of the biological subject being treated, condition of the skin, the duration of the treatment, possible concurrent therapies, the compound, composition or other active ingredient employed in a given formulation, carriers or excipients, if used. As used herein, the term “therapeutically effective amount” may also be referred to herein as a “pharmaceutically effective amount.”

“Thickening agent” as used herein includes an agent or combination of agents that increases the viscosity of a composition. A thickening agent may be a pure substance, or it may comprise, consist essentially of, or consist of a mixture of different chemical entities. Exemplary thickening agents include cellulosic thickening agents, other polysaccharides such as chitosan and the like, carbomer polymers, carbomer derivatives, polyvinyl alcohol, poloxamers, natural gums, as well as mixtures thereof.

“Topical delivery” is used in its conventional sense to mean delivery of an agent, such as a therapeutically active agent, into the viable skin. The outermost layer of the epidermis, the stratum corneum, is lifeless and topical delivery then refers to delivery into the viable epidermis and/or dermis. Topical delivery of a drug may be the advantageous basis, for example, for treatment of various skin disorders. Topical delivery also refers to delivery of an agent into other tissues that are exposed to the environment exterior to the body, such as nail, mucosa or eye.

“Topical formulation” as used herein includes, in one aspect, a composition that is suitable for topical application to the skin, nail, or mucosa. A topical formulation may, for example, be used to confer a therapeutic or cosmetic benefit to its user. Specific topical formulations can be used for superficial, local, regional, or transdermal delivery of substances. The term “topical formulation” as used herein also encompasses the compositions that are formed once a composition that is suitable for topical application to the skin, nail or mucosa is applied to the skin, nail or mucosa. The composition of the topical formulation resulting from such application to the skin, nail or mucosa may differ from the originally applied formulation. For example, components from the originally applied formulation may undergo differential evaporation causing the relative amounts of the ingredients in the formulation to change. Additionally, components from the originally applied formulation may diffuse into the skin, nail or mucosa. Further, compounds that are on or within the substrate to which the formulation is applied may become incorporated into the formulation. For example, linolenic acid and cholesterol, which are natural components of skin, may become incorporated into the formulation.

The term “topical administration” or equivalently “topical application” is used in its conventional sense to mean application of a substance, such as a formulation containing an active agent, to the skin or to a localized more or less external region of the body such as the nail, mucosa or eye. Topical administration may result in any one or more of superficial, topical, regional or transdermal delivery the active agent.

The term “treating” or “treatment” as used herein (and as well understood in the art) means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (e.g., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the patient. The length of the treatment period depends on a variety of factors, such as the severity of the condition, the age and genetic profile of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

As used herein, the term “tolerability” has its ordinary meaning as understood in light of the specification and refers to the extent to which a patient will willingly withstand adverse or side effects of an active compound to achieve the desired therapeutic effect, and can refer to short-term or long-term side effects

As used herein, the terms “efficacy”, “intrinsic activity”, and “potency” have their ordinary meaning as understood in light of the specification and refers to the amount of active pharmaceutical ingredient that is needed to achieve a desired effect.

As used herein, the term “stability” has its ordinary meaning as understood in light of the specification and refers to the ability for the active compound or the formulation containing the active compound to remain effective, intact, and safe for consumption or administration over time under the influence of environmental factors such as temperature, humidity, and light. Stability can be assessed by forced degradation studies according to parameters, conditions, and standards set forth by the Food and Drug Administration (FDA) and International Council for Harmonisation (ICH).

As used herein, the term “dosage forms” has its ordinary meaning as understood in light of the specification and refers to the methods of delivering the active pharmaceutical ingredient and any excipients to an individual. Methods of delivery include but are not limited to oral, pills, tablets, capsules, films, drinks, syrups, powders, pastes, inhalation, aerosol, smoke, vapor, mist, buccal, sublingual, nasal, suppository, parenteral, intradermal, subcutaneous, intramuscular, intraosseous, intraperitoneal, intravenous, topical, cream, gel, liniment, balm, lotion, ointment, liquid drops, or patches. Certain methods of delivery may be preferential to others, for reasons such as comfort to the patient, invasiveness, side effects, level of degradation, e.g. in the digestive tract, level of absorption, onset of action, local vs. general effect, duration of effect, effective dose amount, or therapeutic index.

“Plant product” as used herein refers to leaves, roots, bark, skin, berries, fruit, or any part of, extract of, distillate of, decoction of, abstraction of, concentrate of, essence of, juice of, solution of, tincture of, elixir of, or other form of an active ingredient of a plant.

“Turmeric” as used herein refers to turmeric, turmeric root, or any part of, extract of, distillate of, decoction of, abstraction of, concentrate of, essence of, juice of, solution of, tincture of, elixir of, or other form of an active ingredient of any of the plants in the Curcuma genus such as Curcuma longa. The Curcuma genus includes, but is not limited to, Curcuma aeruginosa, Curcuma albicoma, Curcuma albiflora, Curcuma alismatifolia, Curcuma amada Roxb, Curcuma amarissima, Curcuma angustifolia, Curcuma aromatica, Curcuma attenantiata, Curcuma aurantiuma, Curcuma bhatii, Curcuma burttii, Curcuma caesia, Curcuma ceratotheca, Curcuma chuanezhu, Curcuma chuanhuangjiang, Curcuma chuanyujin, Curcuma cochinchinensis, Curcuma codonantha, Curcuma coerens, Curcuma coriacuma, Curcuma comosa, Curcuma ecomata, Curcuma elata, Curcuma exigua, Curcuma ferruginea, Curcuma flaviflora, Curcuma glans, Curcuma gracillima, Curcuma grandiflora, Curcuma haritha, Curcuma harmandii, Curcuma heyneana, Curcuma inodora, Curcuma inodorata, Curcuma lanceolata, Curcuma larsenii, Curcuma latiflora, Curcuma latifolia, Curcuma leucorrhiza, Curcuma loerzingii, Curcuma longa, Curcuma longispica, Curcuma malabarica, Curcuma meraukensis, Curcuma mutabilis, Curcuma neilgher, Curcuma parvula, Curcuma peethapushpa, Curcuma petiolata, Curcuma phaeocaulis, Curcuma pierreana, Curcuma plicata, Curcuma porphyrotaenia, Curcuma prakasha, Curcuma pseudomontana, Curcuma purpurascens, Curcuma purpurea, Curcuma rapumakata, Curcuma rubrobracteata, Curcuma sattayasaii, Curcuma sichuanensis, Curcuma singularis, Curcuma sparganiifolia, Curcuma stenochila, Curcuma strobilifera, Curcuma sulcata, Curcuma sumatranacu, Curcuma thalakaiensis, Curcuma wenyujin, Curcuma wenchowensis, Curcuma xanthorrhiza, Curcuma yunnanensis, Curcuma zedoaria, and Curcuma zedoaroides.

Compounds in turmeric include, but are not limited to, curcumin, curcuminoids, demethoxycurcumin, bisdemethoxycurcumin, tetrahydrocurcumin, or combinations thereof. Turmeric may be in the form of turmeric root extract, ground turmeric, turmeric powder, turmeric essential oil, turmeric distillate, turmeric hydrosol, turmeric ointment, turmeric tincture, turmeric cream, turmeric liniment, turmeric salve and/or turmeric gel.

“Arnica” as used herein refers to Arnica or any part of, extract of, distillate of, decoction of, abstraction of, concentrate of, essence of, juice of, solution of, tincture of, elixir of, or other form of an active ingredient of any of the plants in the Arnica genus such as Arnica montana. The Arnica genus includes, but is not limited to, Arnica acaulis, Arnica angustifolia, Arnica cernua, Arnica chamissonis, Arnica cordifolia, Arnica dealbata, Arnica discoidea, Arnica fulgens, Arnica gracilis, Arnica griscomii, Arnica intermedia, Arnica lanceolata, Arnica latifolia, Arnica lessingii, Arnica lonchophylla, Arnica longifolia, Arnica louiseana, Arnica mallotopus, Arnica mollis, Arnica montana, Arnica nevadensis, Arnica ovata, Arnica parryi, Arnica porsildiorum, Arnica rydbergii, Arnica sachalinensis, Arnica sororia, Arnica spathulata, Arnica unalaschcensis, Arnica venosa, and Arnica viscosa.

Compounds in Arnica include, but are not limited to, sesquiterpene lactones such as helanin. Arnica may be in the form of Arnica extract, ground Arnica, Arnica powder, Arnica essential oil, Arnica distillate, Arnica hydrosol, Arnica ointment, Arnica tincture, Arnica cream, Arnica liniment, Arnica salve and/or Arnica gel.

“Black pepper” as used herein refers to black pepper or any part of, extract of, distillate of, decoction of, abstraction of, concentrate of, essence of, juice of, solution of, tincture of, elixir of, or other form of an active ingredient of any of the plant Piper nigrum. Compounds in black pepper include, but are not limited to, piperine, tetrahydropiperine, piperic acid, chavicine, isochavicine, isopiperine, piperanine or combinations thereof. Black pepper may be in the form of black pepper extract, ground black pepper, black pepper powder, black pepper essential oil, black pepper distillate, black pepper hydrosol, black pepper ointment, black pepper tincture, black pepper cream, black pepper liniment, black pepper salve and/or black pepper gel. In several embodiments, other peppers or active ingredients thereof, can be used in combination with, or in place of black pepper. For example, in several embodiments, formulations provided for herein comprise capsaicin. However, in several embodiments, the formulations are capsaicin-free.

“Zanthoxylum” as used herein refers to as used herein refers to any part of, extract of, distillate of, decoction of, abstraction of, concentrate of, essence of, juice of, solution of, tincture of, elixir of, or other form of an active ingredient of any of the plants in the Zanthoxylum genus such as Zanthoxylum piperitum. The Zanthoxylum genus includes, but is not limited to, Zanthoxylum acanthopodium, Zanthoxylum aculeatissimum, Zanthoxylum acuminatum, Zanthoxylum ailanthoides, Zanthoxylum albiflorum, Zanthoxylum albuquerquei, Zanthoxylum amamiense, Zanthoxylum amapaense, Zanthoxylum americanum, Zanthoxylum anadenium, Zanthoxylum andamanicum, Zanthoxylum andinum, Zanthoxylum anison, Zanthoxylum anthyllidifolium, Zanthoxylum apiculatum, Zanthoxylum arborescens, Zanthoxylum armatum, Zanthoxylum atchoum, Zanthoxylum austrosinense, Zanthoxylum avicennae, Zanthoxylum backer, Zanthoxylum beechyanum, Zanthoxylum bifoliolatum, Zanthoxylum bissei, Zanthoxylum bonifaziae, Zanthoxylum bouetense, Zanthoxylum brachyacanthum, Zanthoxylum brisasanum, Zanthoxylum buesgenii, Zanthoxylum bungeanum, Zanthoxylum burkillianum, Zanthoxylum calcicole, Zanthoxylum campicola, Zanthoxylum canalense, Zanthoxylum capense, Zanthoxylum caribaeum, Zanthoxylum caudatum, Zanthoxylum celebicum, Zanthoxylum chalybeum, Zanthoxylum chevalieri, Zanthoxylum chuquisaquense, Zanthoxylum ciliatum, Zanthoxylum claessensii, Zanthoxylum clava-herculis, Zanthoxylum coco, Zanthoxylum collinsiae, Zanthoxylum comosum, Zanthoxylum compactum, Zanthoxylum conspersipunctatum, Zanthoxylum cucullatipetalum, Zanthoxylum davyi, Zanthoxylum decaryi, Zanthoxylum delagoense, Zanthoxylum deremense, Zanthoxylum dimorphophyllum, dinklagei, Zanthoxylum dipetalum, Zanthoxylum dissitum, Zanthoxylum diversifolium, Zanthoxylum djalma-batistae, Zanthoxylum domingense, Zanthoxylum dumosum, Zanthoxylum echinocarpum, Zanthoxylum ekmanii, Zanthoxylum eliasii, Zanthoxylum engleri, Zanthoxylum esquirolii, Zanthoxylum fagara, Zanthoxylum fauriei, Zanthoxylum finlaysonianum, Zanthoxylum flavum, Zanthoxylum foliolosum, Zanthoxylum forbesii, Zanthoxylum formiciferum, Zanthoxylum gardneri, Zanthoxylum gentryi, Zanthoxylum ghisbreghtii, Zanthoxylum gillespieanum, Zanthoxylum gilletii, Zanthoxylum glomeratum, Zanthoxylum grandifolium, Zanthoxylum haitiense, Zanthoxylum hamadryadicum, Zanthoxylum harrisii, Zanthoxylum hartii, Zanthoxylum hawaiiense, Zanthoxylum heitzii, Zanthoxylum heterophyllum, Zanthoxylum holtzianum, Zanthoxylum huberi, Zanthoxylum humile, Zanthoxylum impressinervium, Zanthoxylum integrifoliolum, Zanthoxylum iwahigense, Zanthoxylum jamaicense, Zanthoxylum juniperinum, Zanthoxylum kauaense, Zanthoxylum khasianum, Zanthoxylum kleinii, Zanthoxylum kwangsiense, Zanthoxylum laetum, Zanthoxylum laurentii, Zanthoxylum leiboicum, Zanthoxylum lemairei, Zanthoxylum lenticellosum, Zanthoxylum lenticulare, Zanthoxylum lepidopteriphilum, Zanthoxylum leprieurii, Zanthoxylum leratii, Zanthoxylum liboense, Zanthoxylum limoncello, Zanthoxylum lindense, Zanthoxylum macranthum, Zanthoxylum madagascariense, Zanthoxylum magnifasciculatum. Zanthoxylum mananarense, Zanthoxylum mantaro, Zanthoxylum maranionense, Zanthoxylum martinicense, Zanthoxylum mayu, Zanthoxylum megistophyllum, Zanthoxylum melanostictum, Zanthoxylum mezoneurispinosum, Zanthoxylum micranthum, Zanthoxylum mildbraedii, Zanthoxylum minahassae, Zanthoxylum mole, Zanthoxylum mollissimum, Zanthoxylum monogynum, Zanthoxylum motuoense, Zanthoxylum multijugum, Zanthoxylum myriacanthum, Zanthoxylum myrianthum, Zanthoxylum nadeaudii, Zanthoxylum nashi, Zanthoxylum nebuletorum, Zanthoxylum nemorale, Zanthoxylum neocaledonicum, Zanthoxylum nigrum, Zanthoxylum nitidum, Zanthoxylum novoguineense, Zanthoxylum oahuense, Zanthoxylum ovalifolium, Zanthoxylum ovatifoliolatum, Zanthoxylum oxyphyllum, Zanthoxylum panamense, Zanthoxylum pancheri, Zanthoxylum paniculatum, Zanthoxylum paracanthum, Zanthoxylum paulae, Zanthoxylum pentandrum, Zanthoxylum petenense, Zanthoxylum petiolare, Zanthoxylum phyllopterum, Zanthoxylum piasezkii, Zanthoxylum pilosiusculum, Zanthoxylum pilosulum, Zanthoxylum pimpinelloides, Zanthoxylum pinnatum, Zanthoxylum piperitum, Zanthoxylum pluviatile, Zanthoxylum poggei, Zanthoxylum psammophilum, Zanthoxylum pseudoxyphyllum, Zanthoxylum pteracanthum, Zanthoxylum pucro, Zanthoxylum punctatum, Zanthoxylum quassiifolium, Zanthoxylum quinduense, Zanthoxylum renieri, Zanthoxylum retroflexum, Zanthoxylum retusum, Zanthoxylum rhetsa, Zanthoxylum rhodoxylum, Zanthoxylum rhoifolium, Zanthoxylum rhombifoliolatum, Zanthoxylum riedelianum, Zanthoxylum rigidum, Zanthoxylum rubescens, Zanthoxylum sapindifolium, Zanthoxylum sarasinii, Zanthoxylum scandens, Zanthoxylum schinifolium, Zanthoxylum schlechteri, Zanthoxylum schreberi, Zanthoxylum setulosum, Zanthoxylum simulans, Zanthoxylum spinosum, Zanthoxylum spondiifolium, Zanthoxylum sprucei, Zanthoxylum stelligerum, Zanthoxylum stenophyllum, Zanthoxylum subspicatum, Zanthoxylum syncarpum, Zanthoxylum taediosum, Zanthoxylum tahitense, Zanthoxylum tambopatense, Zanthoxylum tetraphyllum, Zanthoxylum tetraspermum, Zanthoxylum thomense, Zanthoxylum thornycroftii, Zanthoxylum thouventii, Zanthoxylum tidorense, Zanthoxylum timoriense, Zanthoxylum tingoassuiba, Zanthoxylum tomentellum, Zanthoxylum tragodes, Zanthoxylum trijugum, Zanthoxylum tsihanimposa, Zanthoxylum undulatifolium, Zanthoxylum unifoliatum, Zanthoxylum usambarense, Zanthoxylum usitatum, Zanthoxylum venosum, Zanthoxylum verrucosum, Zanthoxylum vinkii, Zanthoxylum viride, Zanthoxylum vitiense, Zanthoxylum wutaiense, Zanthoxylum xichouense, Zanthoxylum yakumontanum, Zanthoxylum yuanjiangensis, and Zanthoxylum zanthoxyloides.

Compounds in Zanthoxylum include, but are not limited to, hydroxy-α-sanshool, sesamin, benzophenanthridines, furoquinolines, carbazoles, aporphines, canthinones, acridones, aromatic and aliphatic amides or combinations thereof. Zanthoxylum may be in the form of Zanthoxylum extract, ground Zanthoxylum, Zanthoxylum powder, Zanthoxylum essential oil, Zanthoxylum distillate, Zanthoxylum hydrosol, Zanthoxylum ointment, Zanthoxylum tincture, Zanthoxylum cream, Zanthoxylum liniment, Zanthoxylum salve and/or Zanthoxylum gel.

“Clove” as used herein refers to clove or any part of, extract of, distillate of, decoction of, abstraction of, concentrate of, essence of, juice of, solution of, tincture of, elixir of, or other form of an active ingredient of any of the plant Syzygium aromaticum. Compounds in clove include, but are not limited to, eugenol, acetyl eugenol, beta-caryophyllene, vanillin, crategolic acid, bicornin, gallotannic acid, methyl salicylate, eugenin, kaempferol, rhamnetin, and eugenitin, triterpenoids, oleanolic acid, stigmasterol, campesterol, sesquiterpenes, or combinations thereof. Clove may be in the form of clove extract, ground clove, clove powder, clove essential oil, clove distillate, clove hydrosol, clove ointment, clove tincture, clove cream, clove liniment, clove salve and/or clove gel.

“Thyme” as used herein refers to thyme or any part of, extract of, distillate of, decoction of, abstraction of, concentrate of, essence of, juice of, solution of, tincture of, elixir of, or other form of an active ingredient of any of the plants in the Thymus genus such as Thymus vulgaris. The Thymus genus includes, but is not limited to, Thymus adamovicii, Thymus altaicus, Thymus amurensis, Thymus boissieri, Thymus bracteosus, Thymus broussonetii, Thymus caespititius, Thymus camphoratus, Thymus capitatus, Thymus capitellatus, Thymus camphoratus, Thymus carnosus, Thymus cephalotus, Thymus cherlerioides, Thymus ciliates, Thymus cilicicus, Thymus cimicinus, Thymus citriodorus, Thymus comosus, Thymus comptus, Thymus curtus, Thymus decussatus, Thymus disjunctus, Thymus doerfleri, Thymus dubjanskyi, Thymus glabrescens, Thymus herba-barona, Thymus hirsutus, Thymus hyemalis, Thymus inaequalis, Thymus integer, Thymus lanuginosus, Thymus leucospermus, Thymus leucotrichus, Thymus longicaulis, Thymus longiflorus, Thymus mandschuricus, Thymus marschallianus, Thymus mastichina, Thymus membranaceus, Thymus mongolicus, Thymus moroderi, Thymus nervulosus, Thymus nummularis, Thymus odoratissimus, Thymus pallasianus, Thymus pallidus, Thymus pannonicus, Thymus praecox, Thymus Proximus, Thymus pseudolanuginosus, Thymus pulegioides, Thymus quinquecostatus, Thymus richardii, Thymus satureioides, Thymus serpyllum, Thymus sibthorpii, Thymus striatus, Thymus thracicus, Thymus villosus, Thymus vulgaris, and Thymus zygis.

Compounds in thyme include, but are not limited to, thymol, carvacrol, linalool, α-terpineol, 1,8-cineole, or combinations thereof. Thyme may be in the form of thyme extract, ground thyme, thyme powder, thyme essential oil, thyme distillate, thyme hydrosol, thyme ointment, thyme tincture, thyme cream, thyme liniment, thyme salve and/or thyme gel.

“Piper betle” as used herein refers to Piper betle, Piper betle leaves, or any part of, extract of, distillate of, decoction of, abstraction of, concentrate of, essence of, juice of, solution of, tincture of, elixir of, or other form of an active ingredient of any of the plant Piper betle. Compounds in Piper betle include, but are not limited to, chavibetol, safrole, eugenol, isoeugenol, germacrene D, chavicol, hydroxychavicol, caryophyllene, β-sitosterol, β-daucosterol, stigmasterol, piperine, pellitorine, piperdardine, guineensine, pinoresinol, oleanolic acid, dehydropipernonaline, piperolein-B, bornyl cis-4-hydroxycinnamate, bornyl p-coumarate, aristololactam A-II, 4-allyl resorcinol, stigmast-4-en-3,6-dione, terpinolene, terpinene, cadinene, 3-carene or combinations thereof. Piper betle may be in the form of Piper betle extract, ground Piper betle, Piper betle powder, Piper betle essential oil, Piper betle distillate, Piper betle hydrosol, Piper betle ointment, Piper betle tincture, Piper betle cream, Piper betle liniment, Piper betle salve and/or Piper betle gel.

“Peppermint” as used herein refers to peppermint, Western peppermint, Chinese peppermint, or any part of, extract of, distillate of, decoction of, abstraction of, concentrate of, essence of, juice of, solution of, tincture of, elixir of, or other form of an active ingredient of any of the plant Mentha x piperita, or any part of, extract of, distillate of, decoction of, abstraction of, concentrate of, essence of, juice of, solution of, tincture of, elixir of, or other form of an active ingredient of any of the plant Mentha haplocalyx. Compounds in peppermint include, but are not limited to, menthol, menthone, menthyl acetate, menthofuran, 1,8-cineol, limonene, pulegone, caryophyllene, pinene, eriocitrin, hesperidin, kaempferol 7-O-rutinoside, beta-pinene, beta-caryophyllene, or combinations thereof. Peppermint may be in the form of peppermint extract, ground peppermint, peppermint powder, peppermint essential oil, peppermint distillate, peppermint hydrosol, peppermint ointment, peppermint tincture, peppermint cream, peppermint liniment, peppermint salve and/or peppermint gel.

“Cinchona” as used herein refers to Cinchona, Cinchona bark, or any part of, extract of, distillate of, decoction of, abstraction of, concentrate of, essence of, juice of, solution of, tincture of, elixir of, or other form of an active ingredient of any of the plants in the Cinchona genus such as Cinchona officinalis. The Cinchona genus includes, but is not limited to, Cinchona anderssonii, Cinchona antioquiae, Cinchona asperifolia, Cinchona barbacoensis, Cinchona calisaya, Cinchona capuli, Cinchona fruticosa, Cinchona glandulifera, Cinchona hirsute, Cinchona krauseana, Cinchona lancifolia, Cinchona lucumifolia, Cinchona macrocalyx, Cinchona micrantha, Cinchona mutisii, Cinchona nitida, Cinchona officinalis, Cinchona parabolica, Cinchona pitayensis, Cinchona pubescens, Cinchona pyrifolia, Cinchona rugosa, Cinchona scrobiculata, and Cinchona villosa.

Compounds in cinchona include, but are not limited to cinchonine, cinchonidine, quinine, quinidine, dihydroquinine, dihydroquinidine, and cinchotannic acid, or combinations thereof. Cinchona may be in the form of cinchona extract, ground cinchona, cinchona powder, cinchona essential oil, cinchona distillate, cinchona hydrosol, cinchona ointment, cinchona tincture, cinchona cream, cinchona liniment, cinchona salve and/or cinchona gel.

“Datura” as used herein refers to Datura or any part of, extract of, distillate of, decoction of, abstraction of, concentrate of, essence of, juice of, solution of, tincture of, elixir of, or other form of an active ingredient of any of the plants in the Datura genus such as Datura metel. The Datura genus includes, but is not limited to, Datura arenicola, Datura ceratocaula, Datura discolor, Datura ferox, Datura innoxia, Datura kymatocarpa, Datura lanosa, Datura leichhardtii, Datura metel, Datura pruinosa. Datura quercifolia, Datura reburra, Datura stramonium, and Datura wrightii.

Compounds in Datura include, but are not limited to, scopolamine and atropine, or combinations thereof. Datura may be in the form of Datura extract, ground Datura, datura powder, Datura essential oil, Datura distillate, Datura hydrosol, Datura ointment, Datura tincture, Datura cream, Datura liniment, Datura salve and/or Datura gel.

“Jasmine” as used herein refers to jasmine or any part of, extract of, distillate of, decoction of, abstraction of, concentrate of, essence of, juice of, solution of, tincture of, elixir of, or other form of an active ingredient of any of the plants in the Jasminum genus such as Jasminum officinale. The Jasminum genus includes, but is not limited to, Jasminum abyssinicum, Jasminum adenophyllum, Jasminum andamanicum, Jasminum angulare, Jasminum angustifolium, Jasminum auriculatum, Jasminum azoricum, Jasminum beesianum, Jasminum dichotomum, Jasminum didymium, Jasminum dispermum, Jasminum elegans, Jasminum elongatum, Jasminum floridum, Jasminum fluminense, Jasminum fruticans, Jasminum grandiflorum, Jasminum humile, Jasminum lanceolarium, Jasminum laurifolium, Jasminum malabaricum, Jasminum mesnyi, Jasminum multiflorum, Jasminum multipartitum, Jasminum nervosum, Jasminum nobile, Jasminum nudiflorum, Jasminum odoratissimum, Jasminum officinale, Jasminum parkeri, Jasminum polyanthum, Jasminum sambac, Jasminum simplicifolium, Jasminum sinense, Jasminum subhumile, Jasminum subtriplinerve, Jasminum tortuosum, Jasminum urophyllum, and Jasminum volubile.

Compounds in jasmine include linalool, eugenol, jasmone, jasminaldehyde, ethyljasmonate, jasmonic acid, methyl dihyrdojasmonate, benzyl acetate, or combinations thereof. Jasmine may be in the form of jasmine extract, ground jasmine, jasmine powder, jasmine essential oil, jasmine distillate, jasmine hydrosol, jasmine ointment, jasmine tincture, jasmine cream, jasmine liniment, jasmine salve and/or jasmine gel.

“California sagebrush” as used herein refers to California sagebrush or any part of, extract of, distillate of, decoction of, abstraction of, concentrate of, essence of, juice of, solution of, tincture of, elixir of, or other form of an active ingredient of any of the plant Artemisia californica. Compounds in California sagebrush include camphor, eucalyptol, camphene, β-pinene, borneol, thujone, or combinations thereof, California sagebrush may be in the form of California sagebrush extract, ground California sagebrush, California sagebrush powder, California sagebrush essential oil, California sagebrush distillate, California sagebrush hydrosol, California sagebrush ointment, California sagebrush tincture, California sagebrush cream, California sagebrush liniment, California sagebrush salve and/or California sagebrush gel.

“Lavender” as used herein refers to lavender, or any part of, extract of, distillate of, decoction of, abstraction of, concentrate of, essence of, juice of, solution of, tincture of, elixir of, or other form of an active ingredient of any of the plants in the Lavandula genus such as Lavandula angustifolia. The Lavandula genus include, but is not limited to Lavandula angustifolia, Lavandula latifolia, Lavandula lanata, Lavandula dentata, Lavandula stoechas, Lavandula pedunculata, Lavandula viridis, Lavandula multifida, Lavandula canariensis, Lavandula minutolii, Lavandula bramwellii, Lavandula pinnata, Lavandula buchii, Lavandula rotundifolia, Lavandula maroccana, Lavandula tenuisecta, Lavandula rejdalii, Lavandula mairei, Lavandula coronopifolia, Lavandula saharica, Lavandula antineae, Lavandula pubescens, Lavandula citriodora, Lavandula subnuda, Lavandula macra, Lavandula dhofarensis, Lavandula samhanensis, Lavandula setifera, Lavandula qishnensis, Lavandula nimmoi, Lavandula galgalloensis, Lavandula aristibracteata, Lavandula somaliensis, Lavandula bipinnata, Lavandula gibsonii, Lavandula hasikensis, Lavandula sublepidota, Lavandula atriplicifolia, and Lavandula erythraeae.

Compounds in lavender include, but are not limited to, linalool, linalyl acetate, lavandulyl acetate, terpinene-4-ol, lanvandulol, 1,8-cineole, camphor, limonene, tannins, or combinations thereof. Lavender may be in the form of lavender extract, ground lavender, lavender powder, lavender essential oil, lavender distillate, lavender hydrosol, lavender ointment, lavender tincture, lavender cream, lavender liniment, lavender salve and/or lavender gel.

“Paracress” as used herein refers to paracress, toothache plant, jambu, electric daosy, buzz buttons, Schezuan buttons, or any part of, extract of, distillate of, decoction of, abstraction of, concentrate of, essence of, juice of, solution of, tincture of, elixir of, or other form of an active ingredient of any of the plant Spilanthes acmella, or any part of, extract of, distillate of, decoction of, abstraction of, concentrate of, essence of, juice of, solution of, tincture of, elixir of, or other form of an active ingredient of any of the plant Acmella oleracea. Compounds in paracress include, but are not limited to, spilanthol, (2E,7Z,9E)-Undeca-2,7,9-trienoic acid isobutyl amide, (2E)-Undeca-2-en-8,10-diynoic acid isobutyl amide, stigmasteryl-3-O-b-D-glucopyranoside, triterpenes, or combinations thereof. Paracress may be in the form of paracress extract, ground paracress, paracress powder, paracress essential oil, paracress distillate, paracress hydrosol, paracress ointment, paracress tincture, paracress cream, paracress liniment, paracress salve and/or paracress gel.

“Sterculia” as used herein refers to sterculia gum, gum karaya, Indian gum tragacanth, or any part of, extract of, distillate of, decoction of, abstraction of, concentrate of, essence of, juice of, solution of, tincture of, elixir of, or other form of an active ingredient of any of the plants in the Sterculia genus such as Sterculia tragacantha and Sterculia urens. Compounds in sterculia include, but are not limited to galactose, rhamnose, galacturoic acid, 9,12-Octadecadienoic acid (Z, Z)-, 2-hydroxy-1-(hydroxymethyl) ethyl ester, or combinations thereof. Sterculia may be in the form of sterculia extract, ground sterculia, sterculia powder, sterculia essential oil, sterculia distillate, stericulia hydrosol, sterculia ointment, sterculia tincture, sterculia cream, sterculia liniment, sterculia salve and/or sterculia gel.

“Fennel” as used herein refers to fennel or any part of, extract of, distillate of, decoction of, abstraction of, concentrate of, essence of, juice of, solution of, tincture of, elixir of, or other form of an active ingredient of any of the plant Foeniculum vulgare. Compounds in fennel include, but are not limited to, fenchone, limonene, trans-anethole, estragole, 1-octen-3-ol, rosamarinic acid, luteolin, or combinations thereof. Fennel may be in the form of fennel extract, ground fennel, fennel powder, fennel essential oil, fennel distillate, fennel hydrosol, fennel ointment, fennel tincture, fennel cream, fennel liniment, fennel salve and/or fennel gel.

“Nutmeg” as used herein refers to nutmeg, nutmeg seed, nutmeg spice or any part of, extract of, distillate of, decoction of, abstraction of, concentrate of, essence of, juice of, solution of, tincture of, elixir of, or other form of an active ingredient of any of the plants in the Myristica genus such as Myristica fragrans. Compounds in nutmeg include, but are not limited to, myrsticin, eugenol, D-pinene, limonene, D-borneol, D-terpineol, geraniol, safrole, sabinene, or combinations thereof. Nutmeg may be in the form of nutmeg extract, ground nutmeg, nutmeg powder, nutmeg essential oil, nutmeg distillate, nutmeg hydrosol, nutmeg ointment, nutmeg tincture, nutmeg cream, nutmeg liniment, nutmeg salve and/or nutmeg gel.

“Cinnamon” as used herein refers to cinnamon, cinnamon spice, cinnamon bark, or any part of, extract of, distillate of, decoction of, abstraction of, concentrate of, essence of, juice of, solution of, tincture of, elixir of, or other form of an active ingredient of any of the plants in the Cinnamomum genus such as Cinnamomum verum, Cinnamomum burmanni, Cinnamomum cassia, Cinnamomum loureiroi, and Cinnamomum citriodorum. Compounds in cinnamon include, but are not limited to, cinnamaldehyde, trans-cinnamaldehyde, cinnamyl acetate, eugenol, L-borneol, caryophyllene oxide, b-caryophyllene, L-bornyl acetate, E-nerolidol, α-cubebene, α-terpineol, terpinolene, α-thujene, or combinations thereof. Cinnamon may be in the form of cinnamon extract, ground cinnamon, cinnamon powder, cinnamon essential oil, cinnamon distillate, cinnamon hydrosol, cinnamon ointment, cinnamon tincture, cinnamon cream, cinnamon liniment, cinnamon salve and/or cinnamon gel.

“Basil” as used herein refers to basil, basil leaves, or any part of, extract of, distillate of, decoction of, abstraction of, concentrate of, essence of, juice of, solution of, tincture of, elixir of, or other form of an active ingredient of any of the plant Ocimum basilicum. Compounds in basil include, but are not limited to, linalool, eugenol, methyl eugenol, methyl chavicol, 1-8-cineole, myrcene, or combinations thereof. Basil may be in the form of basil extract, ground basil, basil powder, basil essential oil, basil distillate, basil hydrosol, basil ointment, basil tincture, basil cream, basil liniment, basil salve and/or basil gel.

“Chili pepper” as used herein refers to chili peppers, chili pepper pods, chili pepper seeds, chili pepper skins, chili pepper spice, chili pepper leaves, or any part of, extract of, distillate of, decoction of, abstraction of, concentrate of, essence of, juice of, solution of, tincture of, elixir of, or other form of an active ingredient of any of the plants in the Capsicum genus such as Capsicum annuum. The Capsicum genus includes, but is not limited to Capsicum annuum, Capsicum baccatum, Capsicum benoistii, Capsicum buforum, Capsicum caatingae, Capsicum caballeroi, Capsicum campylopodium, Capsicum carassense, Capsicum cardenasii, Capsicum ceratocalyx, Capsicum chacoense, Capsicum chinense, Capsicum coccineum, Capsicum cornutum, Capsicum dimorphum, Capsicum dusenii, Capsicum eshbaughii, Capsicum eximium, Capsicum flexuosum, Capsicum friburgense, Capsicum frutescens, Capsicum galapagoense, Capsicum geminifolium, Capsicum havanense, Capsicum hookerianum, Capsicum hunzikerianum, Capsicum lanceolatum, Capsicum leptopodum, Capsicum longidentatum, Capsicum longifolium, Capsicum lycianthoides, Capsicum minutiflorum, Capsicum mirabile, Capsicum mositicum, Capsicum neei, Capsicum parvifolium, Capsicum pereirae, Capsicum pubescens, Capsicum piuranum, Capsicum praetermissum, Capsicum ramosissimum, Capsicum recurvatum, Capsicum regale, Capsicum rhomboideum, Capsicum schottianum, Capsicum scolnikianum, Capsicum spina-alba, Capsicum stramoniifolium, Capsicum tovarii, and Capsicum villosum.

Compounds in chili pepper include, but are not limited to, capsaicin, dihydrocapsaicin, nordihydrocapsaicin, homocapsaicin, homodihydrocapsaicin, nonivamine, or combinations thereof. Chili pepper may be in the form of chili pepper extract, ground chili pepper, chili pepper powder, chili pepper essential oil, chili pepper distillate, chili pepper hydrosol, chili pepper ointment, chili pepper tincture, chili pepper cream, chili pepper liniment, chili pepper salve and/or chili pepper gel.

Additionally, in several embodiments, the formulations provided for herein further comprise at least one plant product selected from group consisting of Zanthoxylum, clove, thyme, Piper betle, peppermint, cinchona, Datura, jasmine, California sagebrush, lavender, paracress, sterculia, fennel, nutmeg, cinnamon, basil, and chili pepper. Additionally, in several embodiments, formulations provided for herein comprise Zanthoxylum. However, in several embodiments, the formulations are Zanthoxylum-free. Additionally, in several embodiments, formulations provided for herein comprise clove. However, in several embodiments, the formulations are clove-free. Additionally, in several embodiments, formulations provided for herein comprise thyme. However, in several embodiments, the formulations are thyme-free. Additionally, in several embodiments, formulations provided for herein comprise Piper betle. However, in several embodiments, the formulations are Piper betle-free. Additionally, in several embodiments, formulations provided for herein comprise peppermint. However, in several embodiments, the formulations are peppermint-free. Additionally, in several embodiments, formulations provided for herein comprise cinchona. However, in several embodiments, the formulations are cinchona-free. Additionally, in several embodiments, formulations provided for herein comprise Datura. However, in several embodiments, the formulations are Datura-free. Additionally, in several embodiments, formulations provided for herein comprise jasmine. However, in several embodiments, the formulations are jasmine-free. Additionally, in several embodiments, formulations provided for herein comprise California sagebrush. However, in several embodiments, the formulations are California sagebrush-free. Additionally, in several embodiments, formulations provided for herein comprise lavender. However, in several embodiments, the formulations are lavender-free. Additionally, in several embodiments, formulations provided for herein comprise paracress. However, in several embodiments, the formulations are paracress-free. Additionally, in several embodiments, formulations provided for herein comprise sterculia. However, in several embodiments, the formulations are sterculia-free. Additionally, in several embodiments, formulations provided for herein comprise fennel. However, in several embodiments, the formulations are fennel-free. Additionally, in several embodiments, formulations provided for herein comprise nutmeg. However, in several embodiments, the formulations are nutmeg-free. Additionally, in several embodiments, formulations provided for herein comprise cinnamon. However, in several embodiments, the formulations are cinnamon-free. Additionally, in several embodiments, formulations provided for herein comprise basil. However, in several embodiments, the formulations are basil-free. Additionally, in several embodiments, formulations provided for herein comprise chili pepper. However, in several embodiments, the formulations are chili pepper-free.

Additionally, in several embodiments, formulations provided for herein comprise camphor. However, in several embodiments, the formulations are camphor-free. Additionally, in several embodiments, formulations provided for herein comprise linalool. However, in several embodiments, the formulations are linalool-free. Additionally, in several embodiments, formulations provided for herein comprise menthol. However, in several embodiments, the formulations are menthol-free. Additionally, in several embodiments, formulations provided for herein comprise lidocaine. However, in several embodiments, the formulations are lidocaine-free. Additionally, in several embodiments, formulations provided for herein comprise tetracaine. However, in several embodiments, the formulations are tetracaine-free. Additionally, in several embodiments, formulations provided for herein comprise benzocaine. However, in several embodiments, the formulations are benzocaine-free.

### Formulations

The present application includes topical formulations. Unless otherwise specified, any of, or a plurality of, the compounds within a plant product disclosed herein may be used apart from other compounds within a plant product or each of the compounds disclosed as being in a plant product are used (e.g., the plant product itself is in the formulation).

In some embodiments, the topical formulation comprises Arnica present in an amount of 10% to 25% or about 10% to about 25% w/w; black pepper present in an amount of 0.5% to 7% or about 0.5% to about 7% w/w; and turmeric present in an amount 5% to 16% or about 5% to about 16% w/w.

In some embodiments, the Arnica is in the form of in the form of Arnica extract, ground Arnica, Arnica powder, Arnica essential oil, Arnica distillate, Arnica hydrosol, Arnica ointment, Arnica tincture, Arnica cream, Arnica liniment, Arnica salve and/or Arnica gel. In some embodiments, the Arnica is in the form of Arnica extract. In some embodiments, the black pepper is in the form of black pepper extract, ground black pepper, black pepper powder, black pepper essential oil, black pepper distillate, black pepper hydrosol, black pepper ointment, black pepper tincture, black pepper cream, black pepper liniment, black pepper salve and/or black pepper gel. In some embodiments, the black pepper is in the form of black pepper extract. In some embodiments, the black pepper active ingredient is in the form piperine. In some embodiments, the black pepper active ingredient is in the form chavicine or other piperine isomer. In some embodiments, the turmeric is in the form of in the form of turmeric root extract, ground turmeric, turmeric powder, turmeric essential oil, turmeric distillate, turmeric hydrosol, turmeric ointment, turmeric tincture, turmeric cream, turmeric liniment, turmeric salve and/or turmeric gel. In some embodiments, the turmeric is in the form of turmeric extract.

In some embodiments, the topical formulation also includes a preservative. In some embodiments, the preservative comprises Coenzyme Q10 (CoQ10). In some embodiments, the CoQ10 is present in an amount 0.05% to 1% or about 0.05% to about 1% w/w. Additionally, in several embodiments, formulations provided for herein comprise phenoxyethanol and/or ethylhexylglycerin as preservatives. However, in several embodiments, the formulations are free of one or both of phenoxyethanol and ethylhexylglycerin.

In some embodiments, the Arnica is present in an amount of 13-15% (e.g., 14.6%) or is about 13-15% (e.g., 14.6%) w/w, the black pepper is present in an amount of 0.5-1.5% (e.g., 1.0%) or is about 0.5-1.5% (e.g., about 1.0% w/w), and the turmeric is present in an amount of 7.5-9.0% (e.g., 8.1%) or is about 7.5-9.0% (e.g., about 8.1%) w/w. In several embodiments, when present, CoQ10 is present in an amount of 0.1-0.5% (e.g., 0.3%) or is about 0.1-0.5% (e.g., about 0.3%) w/w.

In some embodiments, the Arnica is present in an amount of 22-25% (e.g., 23.02%) or is about 22-25% (e.g., about 23.02% w/w), the black pepper is present in an amount of 0.5-1.5% (e.g., 0.89%) or is about 0.5-1.5% (e.g., about 0.89%) w/w, and the turmeric extract is present in an amount of 6.5-8% (e.g., 7.3%) or is about 6.5-8% (e.g., about 7.3%) w/w. In several embodiments, when present, CoQ10 is present in an amount of 0.1-0.5% (e.g., 0.26%) or is about 0.1-0.5% (e.g., about 0.26%) w/w.

In some embodiments, the topical formulation comprises at least one plant product selected from group consisting of Zanthoxylum, clove, thyme, Piper betle, peppermint, cinchona, Datura, jasmine, California sagebrush, lavender, paracress, sterculia, fennel, nutmeg, cinnamon, basil, and chili pepper. In some embodiments, the topical formulation further comprises linalool.

In some embodiments, the topical formulation does not comprise a chemical anesthetic. In some embodiments, the topical formulation does not comprise lidocaine, tetracaine, or benzocaine. In some embodiments, the topical formulation does not comprise menthol.

In some embodiments, the topical formulation also includes a pressure sensitive adhesive. In some embodiments, the pressure sensitive adhesive is present in an amount of 48% to 84.5% or about 48% to 84.5% w/w. In some embodiments, the pressure sensitive adhesive is present in an amount of 76.0% or about 76.0% w/w.

### Creams and Serums

In some embodiments, a cream for decreasing soreness, pain, bruising, and/or swelling in a subject comprises a topical formulation of Arnica, black pepper, and turmeric. In some embodiments, the cream also includes a preservative. In some embodiments, the preservative comprises Coenzyme Q10 (CoQ10). In some embodiments, the cream includes any one of the topical formulations disclosed herein. In some embodiments, the soreness, pain, bruising, and/or swelling is localized to an anatomical region of the subject. In some embodiments, the anatomical region comprises skin, nail, mucosa, eye, muscle, and/or bone. In some embodiments, the soreness, pain, bruising, and/or swelling comprises an ecchymosis. In some embodiments, the soreness, pain, bruising, and/or swelling is related to a needle procedure, a surgical procedure, an injury, an insect bite, arthritis, and/or radiation therapy. In some embodiments, the subject is an animal, optionally a human.

In some embodiments, a serum for decreasing soreness, pain, bruising, and/or swelling in a subject comprises a topical formulation of Arnica, black pepper, and turmeric. In some embodiments, the serum also includes a preservative. In some embodiments, the preservative comprises Coenzyme Q10 (CoQ10). In some embodiments, the serum includes any one of the topical formulations disclosed herein. In some embodiments, the soreness, pain, bruising, and/or swelling is localized to an anatomical region of the subject. In some embodiments, the anatomical region comprises skin, nail, mucosa, eye, muscle, and/or bone. In some embodiments, the soreness, pain, bruising, and/or swelling comprises an ecchymosis. In some embodiments, the soreness, pain, bruising, and/or swelling is related to a needle procedure, a surgical procedure, an injury, an insect bite, arthritis, and/or radiation therapy. In some embodiments, the subject is an animal, optionally a human.

### Adhesive Patches

In some embodiments, an adhesive patch for decreasing soreness, pain, bruising, and/or swelling in a subject comprises a first superior surface; a second inferior surface configured for application onto the subject's skin, the second inferior surface comprising a topical formulation, wherein the topical formulation comprises Arnica, black pepper, and turmeric. In some embodiments, the second inferior surface of the adhesive patch further comprises a sterile bandage. In some embodiments, the sterile bandage is located at the center of the second inferior surface of the adhesive patch. In some embodiments, the adhesive patch also includes a preservative. In some embodiments, the preservative comprises Coenzyme Q10 (CoQ10). In some embodiments, the adhesive patch also includes a pressure sensitive adhesive. In some embodiments, the adhesive patch includes any one of the topical formulations disclosed herein. In some embodiments, the adhesive patch further comprises sterile packaging. In some embodiments, the soreness, pain, bruising, and/or swelling is localized to an anatomical region of the subject. In some embodiments, the anatomical region comprises skin, nail, mucosa, eye, muscle, and/or bone. In some embodiments, the soreness, pain, bruising, and/or swelling comprises an ecchymosis. In some embodiments, the soreness, pain, bruising, and/or swelling is related to a needle procedure, a surgical procedure, an injury, an insect bite, arthritis, and/or radiation therapy. In some embodiments, the subject is an animal, optionally a human.

### Kits

In some embodiments, a kit for use in decreasing soreness, pain, bruising, and/or swelling in a subject, comprises a predetermined dose of a topical formulation comprising Arnica, black pepper, and turmeric; and an adhesive dressing. In some embodiments, the kit also includes a preservative. In some embodiments, the preservative comprises Coenzyme Q10 (CoQ10). In some embodiments, the kit includes any one of the topical formulations disclosed herein. In some embodiments, the soreness, pain, bruising, and/or swelling is localized to an anatomical region of the subject. In some embodiments, the anatomical region comprises skin, nail, mucosa, eye, muscle, and/or bone. In some embodiments, the soreness, pain, bruising, and/or swelling comprises an ecchymosis. In some embodiments, the soreness, pain, bruising, and/or swelling is related to a needle procedure, a surgical procedure, an injury, an insect bite, arthritis, and/or radiation therapy. In some embodiments, the subject is an animal, optionally a human.

### Methods of Treatment

In some embodiments, a method for decreasing soreness, pain, bruising, and/or swelling in a subject comprises topical administration to the subject of a therapeutically effective amount of any of the topical formulations disclosed herein.

In some embodiments, the topical formulation is administered to the subject for 8-48 hours or about 8-48 hours.

In some embodiments, the topical formulation is administered to the subject at or around a site of pain. In some embodiments, the site of pain comprises a needle stick site, an injury, a joint, and/or an insect bite. In some embodiments, the topical formulation is administered at or around the needle stick site, post-needle stick.

In some embodiments, the soreness, pain, bruising, and/or swelling is localized to an anatomical region of the subject. In some embodiments, the anatomical region comprises skin, nail, mucosa, eye, muscle, and/or bone. In some embodiments, the soreness, pain, bruising, and/or swelling comprises an ecchymosis. In some embodiments, the soreness, pain, bruising, and/or swelling is related to a needle procedure, a surgical procedure, an injury, an insect bite, arthritis, and/or radiation therapy. In some embodiments, the subject is an animal, optionally a human.

In some embodiments, a method for decreasing soreness, pain, bruising, and/or swelling in a subject comprises applying any of the adhesive patches disclosed herein.

In some embodiments, the adhesive patch is applied to the subject for 8-48 hours or about 8-48 hours.

In some embodiments, the adhesive patch is applied to the subject at or around a site of pain. In some embodiments, the site of pain comprises a needle stick site, an injury, a joint, and/or an insect bite. In some embodiments, the adhesive patch is applied to at or around the needle stick site, post-needle stick.

In some embodiments, the soreness, pain, bruising, and/or swelling is localized to an anatomical region of the subject. In some embodiments, the anatomical region comprises skin, nail, mucosa, eye, muscle, and/or bone. In some embodiments, the soreness, pain, bruising, and/or swelling comprises an ecchymosis. In some embodiments, the soreness, pain, bruising, and/or swelling is related to a needle procedure, a surgical procedure, an injury, an insect bite, arthritis, and/or radiation therapy. In some embodiments, the subject is an animal, optionally a human.

### EXAMPLES

Some of the embodiments discussed above are elaborated upon in further detail in the following examples, which are not in any way intended to limit the scope of the present disclosure. Those in the art will appreciate that many other embodiments that not be specifically discussed in the examples are also provided for herein as described above and in the claims.

### Example 1: Plant-Based Formulation #1 and Post-Procedure Patches

Formulations of topical compositions as provided in Table 1 were prepared. In the topical compositions, Arnica extract is present in an amount of 14.6% or is about 14.6% w/w, black pepper extract is present in an amount of 1.0% or is about 1.0% w/w, turmeric extract is present in an amount of 8.1% or is about 8.1% w/w, CoQ10 present in an amount of 0.3% or is about 0.3% w/w, and the pressure-sensitive adhesive is present in an amount of 76.0% or about 76.0% w/w. Post-procedure patches are manufactured by applying the topical composition applied onto foam with a removable liner. In some embodiments, the post-procedure patches are 1.875 inches in width, 1.875 inches in length, and 3.5 square inches in surface area prior to being die-cut into round shapes. In some embodiments, the post-procedure patches have a rounded pentagon shape with a surface area of around 4 square centimeters.

TABLE 1
Plant-based formulation #1

| | | |
|---|---|---|
| Component | Mg/patch | w/w % |

| | | | |
|---|---|---|---|
| Arnica extract | 16.20 | mg | 14.6% |
| Turmeric root extract | 9.00 | mg | 8.1% |
| Black pepper | 1.10 | mg | 1.0% |

(Cosmoperine ® -
tetrahydopiperine)

| | | | |
|---|---|---|---|
| CoQ10 | 0.32 | mg | 0.3% |

| | |
|---|---|
| Adhesive | 76.0% |

The plant-based post-procedure patch was distributed to 46 subjects. Subjects removed the liner from the post-procedure patch and applied the post-procedure patch at or around the needle stick site, shortly after the needle stick. The subjects kept the post-procedure patch applied to the needle stick site for 8-48 hours prior to removing the post-procedure patch. 14 subjects responded to a feedback survey after using the plant-based post-procedure patch. Subjects reported a reduction in soreness, pain, bruising, and/or swelling of the skin, subcutaneous tissue, and/or muscles at the needle stick site. Subjects scored the plant-based procedure patch: 8.22/10 for pain control, 8.2/10 for help with bruising, and 9.5/10 for help with swelling. 93% of subjects stated that they would use the patch again and 100% or subjects said they would recommend this product to family or friends. 86% of subjects indicated that they would prefer the plant-based procedure patch over a bandage after an injection.

### Example 2: Plant-Based Formulation #2

Formulations of topical compositions as provided in Table 2 are prepared. In the topical compositions, Arnica extract is present in an amount of 23.02% or is about 23.02% w/w, black pepper extract is present in an amount of 0.89% or is about 0.89% w/w, and turmeric extract present in an amount of 7.3% or is about 7.3% w/w, and the CoQ10 present in an amount of 0.26% or is about 0.26% w/w. When applied to a post-needle stick site on the skin of a subject, it is believed that this formulation will result in a reduction in soreness, pain, bruising, and/or swelling of the skin, subcutaneous tissue, and/or muscles at the needle stick site.

TABLE 2
Plant-based formulation #2

| | | |
|---|---|---|
| Component | Mg/patch | w/w % |

| | | | |
|---|---|---|---|
| Arnica extract | 14.02 | mg | 23.02% |
| Turmeric root extract | 4.45 | mg | 7.3% |
| Black pepper (piperine) | 0.54 | mg | 0.89% |
| CoQ10 | 0.16 | mg | 0.26% |

### Formulations as Described Generally Herein

Where percentages are provided for agents, ingredients, and compounds, they can be % m/m, % m/w, % w/w, % m/v, % v/v and variations thereof with respect to the formulation as a whole, unless otherwise indicated.

The use of agents, ingredients and compounds may be used interchangeably herein. In several embodiments, reference to the term “based” includes the recited agent, ingredient or compounds. As used herein, the terms composition and formulation can be used interchangeably. Salt forms of the acids identified herein may be used instead of or in addition to the acid.

With respect to the use of plural and/or singular terms herein, those having skill in the art can translate from the plural to the singular and/or from the singular to the plural as is appropriate to the context and/or application.

As will be understood by one skilled in the art, for any and all purposes, such as in terms of providing a written description, all ranges disclosed herein also encompass any and sub-ranges and combinations of sub-ranges thereof. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth. The phrases “and ranges in between” can include ranges that fall in between the numerical values listed. For example, “1, 2, 3, 10, and ranges in between” can include 1-10, 1-3, 2-10, etc.

While various aspects and embodiments have been disclosed herein, other aspects and embodiments will be apparent to those skilled in the art. The various aspects and embodiments disclosed herein are for purposes of illustration and are not intended to be limiting, with the scope and spirit being indicated by, for example, the following claims.

What is claimed is:

1. A topical formulation comprising:
Arnica present in an amount of 10% to 25% or about 10% to about 25% w/w;
black pepper present in an amount of 0.5% to 7% or about 0.5% to about 7% w/w; and
turmeric present in an amount 5% to 16% or about 5% to about 16% w/w.

2. The topical formulation of claim 1, wherein the Arnica is in the form of in the form of Arnica extract, ground Arnica, Arnica powder, Arnica essential oil, Arnica distillate, Arnica hydrosol, Arnica ointment, Arnica tincture, Arnica cream, Arnica liniment, Arnica salve and/or Arnica gel.

3. The topical formulation of claim 1, wherein the Arnica is in the form of Arnica extract.

4. The topical formulation of claim 1, wherein the black pepper is in the form of black pepper extract, ground black pepper, black pepper powder, black pepper essential oil, black pepper distillate, black pepper hydrosol, black pepper ointment, black pepper tincture, black pepper cream, black pepper liniment, black pepper salve and/or black pepper gel.

5. The topical formulation of claim 1, wherein the black pepper is in the form of piperine or a black pepper extract.

6. The topical formulation of claim 1, wherein the turmeric is in the form of in the form of turmeric root extract, ground turmeric, turmeric powder, turmeric essential oil, turmeric distillate, turmeric hydrosol, turmeric ointment, turmeric tincture, turmeric cream, turmeric liniment, turmeric salve and/or turmeric gel.

7. The topical formulation of claim 1, wherein the turmeric is in the form of turmeric extract.

8. The topical formulation of any claim 1, further comprising a preservative.

9. The topical formulation of claim 8, wherein the preservative comprises Coenzyme Q10 (CoQ10).

10. The topical formulation of claim 9, wherein the CoQ10 is present in an amount 0.05% to 1% or about 0.05% to about 1% w/w.

11. The topical formulation of claim 1, wherein the Arnica is present in an amount of 13-15% or is about 13-15% w/w, the black pepper is present in an amount of 0.5-1.5% or is about 0.5-1.5% w/w, the turmeric is present in an amount of 7.5-9.0% or is about 7.5-9.0% w/w, and the CoQ10 present in an amount of 0.1-0.5% or is about 0.1-0.5% w/w.

12. The topical formulation of claim 1, wherein the Arnica is present in an amount of 22-25% or is about 22-25% w/w, the black pepper is present in an amount of 0.5-1.5% or is about 0.5-1.5% w/w, and the turmeric extract is present in an amount of 6.5-8% or is about 6.5-8% w/w, and the CoQ10 present in an amount of 0.1-0.5% or is about 0.1-0.5% w/w.

13. The topical formulation of any one of claims 1-12, further comprising at least one plant product selected from group consisting of Zanthoxylum, clove, thyme, Piper betle, peppermint, cinchona, Datura, jasmine, California sagebrush, lavender, paracress, sterculia, fennel, nutmeg, cinnamon, basil, and chili pepper.

14. The topical formulation of any one of claims 1-13, further comprising linalool.

15. The topical formulation of any one of claims 1-13, wherein the topical formulation does not comprise a chemical anesthetic.

16. The topical formulation of any one of claims 1-15, wherein the topical formulation does not comprise lidocaine, tetracaine, or benzocaine.

17. The topical formulation of any one of claims 1-16, wherein the topical formulation does not comprise menthol.

18. The topical formulation of any one of claims 1-17, further comprising a pressure sensitive adhesive.

19. The topical formulation of claim 18, wherein the pressure sensitive adhesive is present in an amount of 48% to 84.5% or about 48% to 84.5% w/w.

20. A cream for decreasing soreness, pain, bruising, and/or swelling in a subject, comprising a topical formulation of Arnica, black pepper, and turmeric.

21. The cream of claim 20, further comprising a preservative.

22. The cream of claim 20 or 21, wherein the preservative comprises Coenzyme Q10 (CoQ10).

23. The cream of any one of claims 20-22, wherein the topical formulation is the topical formulation of any one of claims 1-20.

24. The cream of any one of claims 20-23, wherein the soreness, pain, bruising, and/or swelling is localized to an anatomical region of the subject.

25. The cream of claim 24, wherein the anatomical region comprises skin, nail, mucosa, eye, muscle, and/or bone.

26. The cream of any one of claims 20-25, wherein the soreness, pain, bruising, and/or swelling comprises an ecchymosis.

27. The cream of any one of claims 20-26, wherein the soreness, pain, bruising, and/or swelling is related to a needle procedure, a surgical procedure, an injury, an insect bite, arthritis, and/or radiation therapy.

28. The cream of any one of claims 20-27, wherein the subject is an animal, optionally a human.

29. A serum for decreasing soreness, pain, bruising, and/or swelling in a subject, comprising a topical formulation of Arnica, black pepper, and turmeric.

30. The serum of claim 29, further comprising a preservative.

31. The serum of claim 29 or 30, wherein the preservative comprises Coenzyme Q10 (CoQ10).

32. The serum of any one of claims 29-31, wherein the topical formulation is the topical formulation of any one of claims 1-19.

33. The serum of any one of claims 29-32, wherein the soreness, pain, bruising, and/or swelling is localized to an anatomical region of the subject.

34. The serum of claim 33, wherein the anatomical region comprises skin, nail, mucosa, eye, muscle, and/or bone.

35. The serum of any one of claims 29-34, wherein the soreness, pain, bruising, and/or swelling comprises an ecchymosis.

36. The serum of any one of claims 29-35, wherein the soreness, pain, bruising, and/or swelling is related to a needle procedure, a surgical procedure, an injury, an insect bite, arthritis, and/or radiation therapy.

37. The serum of any one of claims 29-36, wherein the subject is an animal, optionally a human.

38. An adhesive patch for decreasing soreness, pain, bruising, and/or swelling in a subject, comprising:
a first superior surface; and
a second inferior surface configured for application onto the subject's skin, the second inferior surface comprising a topical formulation, wherein the topical formulation comprises Arnica, black pepper, and turmeric.

39. The adhesive patch of claim 38, wherein the second inferior surface further comprises a sterile bandage.

40. The adhesive patch of claim 39, wherein the sterile bandage is located at the center of the second inferior surface.

41. The adhesive patch of any one of claims 38-40, further comprising a preservative.

42. The adhesive patch of any one of claims 38-41, wherein the preservative comprises Coenzyme Q10 (CoQ10).

43. The adhesive patch of any one of claims 38-42, further comprising a pressure sensitive adhesive.

44. The adhesive patch of any one of claims 38-43, further comprising the topical formulation of any one of claims 1-19.

45. The adhesive patch of any one of claims 38-44, further comprising sterile packaging.

46. The adhesive patch of any one of claims 38-45, wherein the soreness, pain, bruising, and/or swelling is localized to an anatomical region of the subject.

47. The adhesive patch of claim 46, wherein the anatomical region comprises skin, nail, mucosa, eye, muscle, and/or bone.

48. The adhesive patch of any one of claims 38-47, wherein the soreness, pain, bruising, and/or swelling comprises an ecchymosis.

49. The adhesive patch of any one of claims 38-48, wherein the soreness, pain, bruising, and/or swelling is related to a needle procedure, a surgical procedure, an injury, an insect bite, arthritis, and/or radiation therapy.

50. The adhesive patch of any one of claims 38-49, wherein the subject is an animal, optionally a human.

51. A kit for use in decreasing soreness, pain, bruising, and/or swelling in a subject, the kit comprising:
(i) a predetermined dose of a topical formulation comprising Arnica, black pepper, and turmeric; and
(ii) an adhesive dressing.

52. The kit of claim 51, wherein the topical formulation further comprising a preservative.

53. The kit of claim 51 or 52, wherein the preservative comprises Coenzyme Q10 (CoQ10).

54. The kit of any one of claims 52-54, wherein the topical formulation is topical formulation of any one of claims 1-19.

55. The kit of any one of claims 51-54, wherein the soreness, pain, bruising, and/or swelling is localized to an anatomical region of the subject.

56. The kit of any one of claim 55, wherein the anatomical region comprises skin, nail, mucosa, eye, muscle, and/or bone.

57. The kit of any one of claims 51-56, wherein the soreness, pain, bruising, and/or swelling comprises an ecchymosis.

58. The kit of any one of claims 51-57, wherein the soreness, pain, bruising, and/or swelling is related to a needle procedure, a surgical procedure, an injury, an insect bite, arthritis, and/or radiation therapy.

59. The kit of any one of claims 51-58, wherein the subject is an animal, optionally a human.

60. A method for decreasing soreness, pain, bruising, and/or swelling in a subject, comprising topical administration to the subject of a therapeutically effective amount of the topical formulation of any one of claims 1-19.

61. The method of claim 60, wherein the topical formulation is administered to the subject for 8-48 hours or about 8-48 hours.

62. The method of claim 60 or 61, wherein the topical formulation is administered to the subject at or around a site of pain.

63. The method of claim 62, wherein the site of pain comprises a needle stick site, an injury, a joint, and/or an insect bite.

64. The method of claim 63, wherein the topical formulation is administered at or around the needle stick site, post-needle stick.

65. The method of any one of claims 60-64, wherein the soreness, pain, bruising, and/or swelling is localized to an anatomical region of the subject.

66. The method of claim 65, wherein the anatomical region comprises skin, nail, mucosa, eye, muscle, and/or bone.

67. The method of any one of claims 60-66, wherein the soreness, pain, bruising, and/or swelling comprises an ecchymosis.

68. The method of any one of claims 60-67, wherein the soreness, pain, bruising, and/or swelling is related to a needle procedure, a surgical procedure, an injury, an insect bite, arthritis, and/or radiation therapy.

69. The method of any one of claims 60-68, wherein the subject is an animal, optionally a human.

70. A method for decreasing soreness, pain, bruising, and/or swelling in a subject, comprising applying the adhesive patch of any one of claims 38-50 to the subject.

71. The method of claim 70, wherein the adhesive patch is applied to the subject for 8-48 hours or about 8-48 hours.

72. The method of claim 70 or 71, wherein the adhesive patch is applied to the subject at or around a site of pain.

73. The method of claim 72, wherein the site of pain comprises a needle stick site, an injury, a joint, and/or an insect bite.

74. The method of claim 73, wherein the adhesive patch is applied to at or around the needle stick site, post-needle stick.

75. The method of any one of claims 70-74, wherein the soreness, pain, bruising, and/or swelling is localized to an anatomical region of the subject.

76. The method of claim 75, wherein the anatomical region comprises skin, nail, mucosa, eye, muscle, and/or bone.

77. The method of any one of claims 70-76, wherein the soreness, pain, bruising, and/or swelling comprises an ecchymosis.

78. The method of any one of claims 70-77, wherein the soreness, pain, bruising, and/or swelling is related to a needle procedure, a surgical procedure, an injury, an insect bite, arthritis, and/or radiation therapy.

79. The method of any one of claims 70-78, wherein the subject is an animal, optionally a human.
